SEQUENCE LISTING (14 sequences)

SEQ ID NO: 1 - DNA, 22 bp, Artificial Sequence
  primer FP1F-5
catatggcgaaaccgagctatc

SEQ ID NO: 2 - DNA, 26 bp, Artificial Sequence
  primer FP1F-3
ccatggtttgtatgtcggcgggtaag

SEQ ID NO: 3 - DNA, 24 bp, Artificial Sequence
  primer FP3-5
ccatgggcggattattatggcccg

SEQ ID NO: 4 - DNA, 26 bp, Artificial Sequence
  primer FP3-3
ggatccataatatttacggccccaac

SEQ ID NO: 5 - DNA, 26 bp, Artificial Sequence
  primer FP1R-5
ggatccgccaaaccttcttacccacc

SEQ ID NO: 6 - DNA, 32 bp, Artificial Sequence
  primer FP1R-3
ctcgagtcaaagcttgtacgttggaggataag

SEQ ID NO: 7 - DNA, 519 bp, Artificial Sequence
  fp-131 coding sequence
atggcgaaaccgagctatccgccgacctataaagcaaagccgtcttatccaccgacctacaaggcgaaaccaagctatccaccaacctataaggcgaaaccatcttatccgccaacctacaaagccaagccaagctacccgccaacatataaagccaaaccgtcttacccgccgacatacaaaccatgggcggattattatggcccgaaatatggcccgccgcgtcgttatggtggcggaaactataaccgttatggccgtcgttatggtggatataaaggctggaacaacggctggaaacgtggccgttggggccgtaaatattatggatccgccaaaccttcttacccaccgacatataaggccaagccgagctacccaccaacatacaaggcaaaaccttcctatccacctacgtataaagcgaaacctagctatcctccgacgtacaaagcgaagccgtcctatccgcctacgtataaggcgaagccttcttatcctccaacgtacaagctttga

SEQ ID NO: 8 - protein, 172 aa, Artificial Sequence
  fp131 amino acid sequence
MAKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 9 - DNA, 138 bp, Artificial Sequence
  fp-3 variant A
gcggattattatggcccgaaatatggcccgccgcgtcgttatggtggcggaaactataaccgttatggccgtcgttatggtggatataaaggctggaacaacggctggaaacgtggccgttggggccgtaaatattat

SEQ ID NO: 10 - protein, 46 aa, Artificial Sequence
  fp-3 variant A
ADYYGPKYGPPRRYGGGNYNRYGRRYGGYKGWNNGWKRGRWGRKYY

SEQ ID NO: 11 - DNA, 591 bp, Artificial Sequence
  fp-151
atggcgaaaccgagctatccgccgacctataaagcaaagccgtcttatccaccgacctacaaggcgaaaccaagctatccaccaacctataaggcgaaaccatcttatccgccaacctacaaagccaagccaagctacccgccaacatataaagccaaaccgtcttacccgccgacatacaaaagctctgaagaatataaaggcggctattatccgggcaacacctaccattatcattctggcggcagctatcatggctctggctatcatggcggctataaaggcaaatattatggcaaagcgaaaaaatattattataaatataaaaacagcggcaaatataaatatctgaaaaaagcgagaaaatatcatagaaaaggctataaaaaatattatggcggcagcagcgccaaaccttcttacccaccgacatataaggccaagccgagctacccaccaacatacaaggcaaaaccttcctatccacctacgtataaagcgaaacctagctatcctccgacgtacaaagcgaagccgtcctatccgcctacgtataaggcgaagccttcttatcctccaacgtacaagtga

SEQ ID NO: 12 - DNA, 594 bp, Artificial Sequence
  fp-151-3.2 (codon optimization)
atggcgaaaccgagctatccgccgacctataaagcaaagccgtcttatccaccgacctacaaggcgaaaccaagctatccaccaacctataaggcgaaaccatcttatccgccaacctacaaagccaagccaagctacccgccaacatataaagccaaaccgtcttacccgccgacatacaaaagctctgaagaatataaaggcggctattatccgggcaacacctaccattatcattctggcggcagctatcatggctctggctatcatggcggctataaaggcaaatattatggcaaagcgaaaaaatattattataaatataaaaacagcggcaaatataaatatctgaaaaaagcgagaaaatatcatagaaaaggctataaaaaatattatggcggcagcagcgccaaaccttcttacccaccgacatataaggccaagccgagctacccaccaacatacaaggcaaaaccttcctatccacctacgtataaagcgaaacctagctatcctccgacgtacaaagcgaagccgtcctatccgcctacgtataaggcgaagccttcttatcctccaacgtacaagctttga

SEQ ID NO: 13 - protein, 197 aa, Artificial Sequence
  fp-151-3.2 (codon optimization)
MAKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGSSAKPSYPPTYKAKPSYPPTYKAKPSYPPTYKAKPSYPPTYKAKPSYPPTYKAKPSYPPTYKL

SEQ ID NO: 14 - protein, 175 aa, Artificial Sequence
  fp-353
MADYYGPKYGPPRRYGGGNYNRYGRRYGGYKGWNNGWKRGRWGRKYYEFSSEEYKGGYYPGNSNHYHSGGSYHGSGYHGGYKGKYYGKAKKYYYKYKNSGKYKYLKKARKYHRKGYKKYYGGGSSKLADYYGPKYGPPRRYGGGNYNRYGRRYGGYKGWNNGWKRGRWGRKYYLE

(19) United States
(12) Patent Application Publication
(10) Pub. No.: US 2012/0202748 A1
(43) Pub. Date: Aug. 9, 2012

(54) RECOMBINANT MUSSEL ADHESIVE PROTEIN FP-131

(75) Inventors: Hyung Joon CHA, Pohang (KR); Dong Gyun KANG, Gangneung (KR); Young Hoon SONG, Pohang (KR); Yoo Seong CHOI, Seoul (KR); Seonghye LIM, Daejeon (KR)

(73) Assignee: POSTECH ACADEMY-INDUSTRY FOUNDATION, Pohang (KR)

(21) Appl. No.: 13/367,821

(22) Filed: Feb. 7, 2012

Related U.S. Application Data
(60) Provisional application No. 61/439,962, filed on Feb. 7, 2011.

(51) Int. Cl. A61K 38/16; C07H 21/04; C09J 189/00; C12N 5/10; C12P 21/06; A61P 17/02; C07K 14/435; C12N 15/63

(52) U.S. Cl. 514/19.1; 530/350; 536/23.1; 435/320.1; 435/325; 435/419; 435/348; 435/69.1; 106/124.4

(57) ABSTRACT

The present invention relates to a bio-adhesive derived from mussel. In particular, it relates to a recombinant protein fp(foot protein)-131 that is a hybrid of fp-3 variant A and fp-1. According to the present invention, the recombinant protein with adhesive activity can be economically produced in large scale to be used in place of chemical adhesives.

### CROSS-REFERENCE TO RELATED APPLICATION

This application claims priority to and the benefit of Provisional Application No. 61/439,962 filed on Feb. 7, 2011, the entire contents of which are incorporated herein by reference.

### BACKGROUND OF THE INVENTION

1. Field of the Invention

The present invention relates to a bio-adhesive derived from mussel, and more particularly to a recombinant protein fp(foot protein)-131 that is a hybrid of fp-3 variant A and fp-1.

2. Background of the Invention

Marine mussels produce and secrete adhesive proteins that allow them to tightly attach themselves to wet solid surfaces such as underwater rocks, and thus fight tidal currents or buoyancy in the aqueous saline environment. This strong and water-insoluble adhesion has attracted interest for potential use in biotechnological applications.

In addition, mussel adhesive proteins can also be used as medical adhesives as they are non-toxic to the human body and do not impose immunogenicity (Dove et al., Journal of American Dental Association. 112:879, 1986). Moreover, their biodegradable properties make them environmentally friendly.

The byssus can be divided into distal and proximal parts. The proximal part is connected to the stem gland of the mussel foot, while the distal part is connected to the adhesive plaques. The adhesive plaque is composed of five distinct types of proteins: foot protein type 1 (fp-1) to type 5 (fp-5) (Deming, T. J., Current Opinion in Chemical Biology. 3:100-105, 1999).

All of the mussel adhesive proteins contain high ratios of 3,4-dihydroxyphenyl-L-alanine (DOPA), which is derived from hydroxylation of tyrosine residues (Waite, J. H., Biology Review. 58:209-231, 1983). The adhesive proteins closest to the adhesion interface have the highest proportion of DOPA residues (Waite, J. H., Integr. Comp. Biol. 42:1172-1180, 2002). In contrast, mussel adhesive protein analogs lacking DOPA show greatly reduced adhesion abilities (Yu et al., Journal of American Chemical Society. 121:5825-5826, 1999). Indeed, a biochemical study showed that DOPA residues can enable mussel adhesive protein molecules to cross-link with each other via oxidative conversion to DOPA o-quinone. Thus, the DOPA content of a mussel adhesive protein appears to be specifically related to its adhesive properties.

Currently Cell-Tak, a naturally extracted mussel adhesive protein product, is commercially available. This adhesive is mainly composed of fp-1 and fp-2 type proteins, with a minor portion of fp-3. However, the natural extraction process is labor-intensive and inefficient, requiring around 10,000 mussels for 1 g of protein (Morgan, D., The Scientist. 4:1-6, 1990).

Therefore, researchers have sought to produce recombinant mussel adhesive proteins, for example fp-1, in expression systems such as Escherichia coli and yeast. However, these previous studies failed to express functional and economical mussel adhesive proteins due to a number of complications, including a highly biased amino acid composition (5 amino acid types comprise about 89% of the total amino acids in fp-1), different codon usage preferences between mussel and other expression systems (tRNA utilization problems) and low protein yields (U.S. Pat. No. 5,242,808; Filpula et al., Biotechnol. Prog. 6:171-177, 1990; Salerno et al., Applied Microbiology and Biotechnology 58:209-214, 1993; Kitamura et al., Journal of Polymer Science Part A: Polymer Chemistry, 37:729-736, 1999).

To overcome the problems of low-level production and poor purification yield of natural mussel adhesive proteins, and thus to expand the practical applications of mussel adhesive proteins, previously we successfully produced recombinant hybrid fp proteins, fp-151 which is composed of six fp-1 decapeptide repeats at both termini of fp-5 (WO2005/092920), and fp-353 which is composed of fp-3 variant A at both termini of fp-5 (WO2006/107183). However, fp-151 and fp-353 have a necessity of improving solubility and adhesion force.

### SUMMARY OF THE INVENTION

To overcome the aforementioned problems in the prior art, an objective of the present invention is to provide a novel recombinant adhesive protein derived from a mussel.

The present invention provides an isolated adhesive protein fp-131 comprising SEQ ID NO:8.

Preferably, the adhesive protein may further comprises a peptide for improving a physicochemical property selected from the group consisting of solubility, adhesion force, cross-linking, and improvement in protein expression, purification, recovery rate, and biodegradability of the adhesive protein, and the peptide is attached to a carboxy-termini or amino-termini of the adhesive protein comprising SEQ ID NO:8. More preferably, the peptide may be isolated from an adhesive protein, and the adhesive protein may be isolated from a mussel adhesive protein.

The present invention also provides an adhesive comprising the adhesive protein as an active component.

Preferably, 5% to 100% of the total number of tyrosine residues in the adhesive protein may be modified to 3,4-dihydroxyphenyl-L-alanine (DOPA).

Preferably, the adhesive may adheres to a substrate selected from the group consisting of plastic, glass, metal, eukaryotic cells, prokaryotic cells, and plant cell walls and lipids.

Preferably, the adhesive may be applied to biological sample.

Preferably, the adhesive may further comprises one or more material selected from the group consisting of surfactant, oxidant, and filler. The filler may be selected from the group consisting of collagen, hyaluronic acid, condroitan sulfate, elastine, laminin, caseine, hydroxyapatite, and albumin, fibronectin, and hybrin.

Preferably, the adhesive may be applied to substrates used in an underwater environment.

The present invention also provides a coating material containing the adhesive protein as an active component.

The present invention also provides a polynucleotide comprising a nucleotide sequence encoding the isolated adhesive protein.

Preferably, the nucleotide sequence encoding the adhesive protein comprises a nucleotide sequence comprising SEQ ID NO:7.

Preferably, wherein the nucleotide sequence encoding the adhesive protein may further comprises a nucleotide sequence encoding a peptide for improving a physicochemical property selected from the group consisting of solubility, adhesion force, cross-linking, and improvement in protein expression, purification, recovery rate, and biodegradability of the adhesive protein, and the peptide is attached to a carboxy- and/or amino-termini of the adhesive protein comprising SEQ ID NO:8.

The present invention also provides a vector that comprises a nucleotide sequence encoding the adhesive protein.

The present invention also provides a transformant transformed with the vector.

The present invention also provides a method of producing the adhesive protein comprising the steps of:

(a) constructing a vector that comprises a nucleotide sequence encoding the adhesive protein comprising SEQ ID NO:8;

(b) constructing a transformant by transforming the vector into a host cell; and

(c) producing recombinant adhesive protein by culturing the transformant and purifying the recombinant adhesive protein.

### BRIEF DESCRIPTION OF THE DRAWINGS

FIG. 1 shows major component of the recombinant vector for expression of fp-151 and fp-131.

FIG. 2 shows the result of electrophoresis on a SDS-polyacrylamide gel of purified fp-151 and fp-131.

FIG. 3 shows the result of measuring the water solubility of fp-151 and fp-131. The freeze-dried mussel adhesive proteins fp-151 and fp-131 were dissolved in a 50 mM Tris-HCl (pH6.2) buffer solution at a concentration of 500 mg/mL, and quantified to analyze the solubility.

FIG. 4 shows the result of measuring the bulk-scale adhesion force of fp-151 and fp-131 in wood samples.

FIG. 5 shows the result of measuring the bulk-scale adhesion force of fp-353 and fp-131 in leather samples.

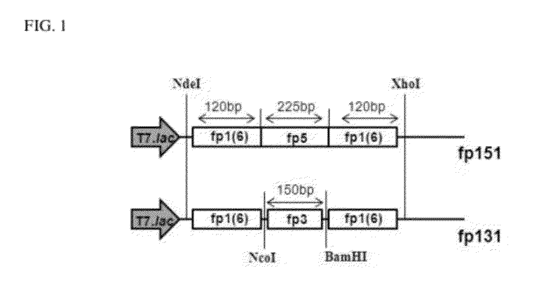

### DETAILED DESCRIPTION OF THE PREFERRED EMBODIMENTS

The present invention relates to a bio-adhesive derived from mussel, and more particularly to a recombinant protein fp(foot protein)-131 that is a hybrid of fp-3 variant A and fp-1.

The adhesive protein of the present invention has the characteristic of attaching to a wide variety of substrates such as glass, metal, polymer resin, plastic or biological cell membranes such as prokaryotic membranes, eukaryotic membranes, and plant cell walls and lipids.

The adhesive protein of the present invention has at least 50% homology with the amino acid sequence shown in SEQ ID NO: 8, preferably 80%, more preferably 90%, and most preferably at least 95% homology, and at the same time can include amino acid sequences that have adhesive property, for example adhesive property that is similar to the amino acid sequence shown in SEQ ID NO: 8, or amino acid sequences that have 70 to 200% of the adhesive activity of the above. For example, there is a protein that contains the amino acid sequence shown in SEQ ID NO: 8. An adhesive protein that contains the amino acid sequence as shown in the above SEQ ID NO: 8 is referred to as “fp-131” from here on.

A nucleotide encoding fp-131 can be expressed as a variety of nucleotide sequences depending on the amino acid codon usage, such as the nucleotide sequences shown in SEQ ID NO: 7.

Also, the adhesive protein of the present invention can further contain a peptide at the amino- and/or carboxy-termini in order to improve the physicochemical properties of the adhesive protein. The above peptide may be added for the purpose of improving for example, the solubility, adhesion force, degree of crosslinking, and expression, purification, recovery rate and biodegradability of the adhesive protein.

The above peptide preferably contains an amino acid sequence derived from an adhesive protein, and more preferably contains an amino acid sequence derived from a mussel adhesive protein.

The adhesive protein and recombinant adhesive protein of the present invention can be inserted into commonly used expression vectors constructed for expressing exogenous genes, and mass-produced through genetic engineering methods. The above vector may be selected according to the type and characteristics of the host cell used in the production of protein, or it may be newly constructed. Transforming the vector into the host cell and producing the recombinant protein from the transformant can easily be carried out through ordinarily employed methods. Selecting, constructing, transforming the vector and expressing the recombinant protein can be easily carried out by an ordinary person skilled in the art of the present invention, and partial variations in the ordinarily employed methods are also included in the present invention.

The sequence encoding an adhesive protein that is inserted into the vector is a sequence encoding an adhesive protein or a recombinant adhesive protein of the present invention, and is preferably selected from the group consisting of a nucleic acid encoding a protein that has at least 50% homology, preferably 80%, more preferably 90%, and most preferably at least 95% homology with the amino acid sequence shown in SEQ ID NO: 8, a nucleic acid encoding a protein that has at least 50% homology, preferably 80%, more preferably 90%, and most preferably at least 95% homology with the amino acid sequence shown in SEQ ID NO: 8.

The expression vector for the adhesive protein and recombinant adhesive protein can be transformed into a host cell selected from the group consisting of prokaryotes, eukaryotes, and eukaryote-derived cells, in order to construct a transformant. The prokaryote is selected from the group consisting of E. coli and Bacillus, the eukaryote is selected from the group consisting of yeast, insects, animals, and plants, and the eukaryote-derived cells are plant cells, insect cells, and mammalian cells, but is not limited thereto.

In an embodiment of the present invention, a pFP131 that expresses a recombinant protein having a structure of 6×AKPSYPPTYK-fp-3 variant A-6×AKPSYPPTYK was constructed. The pFP131 was transformed into E. coli BL21(DE3), to construct E coli BL21/pFP131. The aforementioned transformant can be cultured in typical LB media, and IPTG can be added to induce protein expression. The preferred method of expression of recombinant protein is to culture in LB media (5 g/liter yeast extract, 10 g/liter Tryptone, 10 g/liter NaCl), and adding 0.1 to 10 mM of IPTG when the optical density of the culture solution is 0.6 to 0.9 at 600 nm, then culturing for 2 to 12 hours.

The recombinant protein expressed in the above method is expressed in a water-soluble and/or insoluble form within the transformant, so the isolation and purification depends on how it is expressed. When it is expressed in a water-soluble form, the recombinant protein can be purified by running the lysed cell supernatant through a chromatography column filled with an affinity resin such as a nickel resin. When it is expressed in a water-insoluble form, the recombinant protein can be purified by suspending the lysed cell pellet in an acidic organic solvent, preferably an organic solvent with a pH of 3 to 6, then centrifuging the suspension to isolate the upper layer. Examples of the acidic organic solvent are acetic acid, citric acid, and lactic acid, but is not limited thereto. The acetic acid used can be 5 to 30 (v/v) %, and preferably the cell pellet is dissolved in 20 to 30 (v/v) % acetic acid solution. The upper layer obtained through treatment with acidic organic solvent can further undergo gel filtration chromatography to further purify the recombinant protein.

Through the method of the present invention, the recombinant adhesive protein fp-131 of at least 95% purity can be obtained. The solubility of fp-131 is significantly higher compared to fp-353 and fp-151, and thus fp-131 is easier to obtain in a concentrated form. The solubility of an adhesive protein is directly related to its ability to stay in highly concentrated forms, so the higher the solubility, the easier it is to make highly concentrated forms with high potential for industrial application. In this respect, it can be said that the adhesive protein fp-131 is more useful than fp-353 and fp-151.

The adhesive protein and the recombinant adhesive protein obtained through its expression in the present invention have adhesive activity and can be used as adhesives. The adhesive activity was confirmed through the experiment of modifying the tyrosine residues in the protein to 3,4-dihydroxyphenyl-L-alanine (DOPA). Thus, the adhesive protein of the present invention can not only be used as an adhesive for a wide variety of substrates, but also be used as a bioadhesive since it is harmless to the human body.

The present invention also provides an adhesive that contains adhesive protein as an active component. The adhesive protein can be a form where 5 to 100% of its tyrosine residues are modified to DOPA, and the adhesive can additionally contain a substance that modifies the tyrosine residues in the protein to DOPA. A typical example of the above substance is tyrosinase, but is not limited thereto.

The above adhesive can further contain 0.5 to 90% by weight of an excipient that is generally contained in bioadhesives or is pharmaceutically acceptable. Examples of excipients include surfactants, oxidants, and fillers, but are not limited thereto (see: US Pat. Application Publication No. 2003-65060 and U.S. Pat. No. 5,015,677). The surfactant can be cationic, anionic, non-ionic, or amphoteric, where examples are sodium dodecylsulfate and sodium dodecylbenzensulfonate. The oxidant can be selected from the group consisting of tyrosinase, catechol oxidase, glutaraldehyde, formaldehyde, bis(sulfosuccinimidyl)suberate, 3,3′-Dithiobis(sulfosuccinimidyl propionate), O₂, Fe³⁺, H₂O₂ and IO₄⁻ (see: Macromolecules 1998, 31, 4739-4745), and the filler can be selected from the group consisting of collagen, hyaluronic acid, condroitan sulfate, elastine, laminin, caseine, hydroxyapatite, albumin, fibronectin, and hybrin.

The adhesive of the present invention can be used to adhere or fix glass, plastic, polymer resin, or biological specimen, and the detailed mode and amount of usage, formulation and other such matters may follow Cell-Tak (BD Biosciences, Two Oak Park, Bedford, Mass., USA) which is currently available commercially. For example, the adhesive of the present invention can be a soluble, water-soluble, or insoluble formulation, and can be used in the amount of 0.01 to 100 ug/cm² for a substrate but is not limited thereto. Furthermore, the mode of use follows the general mode of adhesive use, and the typical mode is coating.

The aforementioned biological specimen refers to any animal or plant categorized as a biological organism and any part derived from such animal or plant. For example, it refers to cells, tissues, organs, RNA, DNA, protein, peptide, polynucleotide, hormones, and compounds, but is not limited thereto.

Examples of application of the adhesive of the present invention are as follows, but not limited thereto: (1) adhesion of substrates under water (fresh or salt water); (2) orthopedic treatments such as treatment of bone, ligament, tendon, meniscus, and muscle, and implant of artificial materials; (3) treatment of perforations, lacerations, and cuts, and ophthalmic attachments such as corneal implants and artificial corneal implants; (4) dental attachments such as holding retainers, bridges, or crowns in place, securing loose teeth, repairing broken teeth, and holding fillers in place; (5) surgical treatments such as attachment of blood vessels, attachment of cellular tissue, artificial material implants, and closure of wounds; (6) plant attachments such as bonding of transplanted parts and wound healing; (7) drugs, hormones, biological factors, medications, physiological or metabolic monitoring equipment, antibiotics, and cell transplant (see: U.S. Pat. No. 5,015,677).

The present invention also provides a method of adjusting the adhesion force of the above adhesive by treating with a substance selected from the group consisting of surfactant, oxidant, and filler, or controlling the concentration of the adhesive protein which is an active component of the adhesive (see: U.S. Pat. No. 5,015,677). The surfactant, oxidant, and filler are the same as was described above.

The present invention also provides a coating agent which contains the above adhesive protein as an active component. Since the adhesive protein of the present invention has the characteristic of adhering to glass, plastic, metal, polymer resin, or biological specimen such as eukaryotic cells, prokaryotic cells, plant cell walls and lipids, it can not only be used as a coating agent for these substrates, but also coat the surface of substrates that are used underwater to prevent oxidation of the substrates, since the adhesive protein is water-resistant and water-repellent. An example of application of the coating agent is to coat the motor propeller of ships to prevent corrosion, but is not limited thereto. The above coating agent may consist solely of an adhesion protein, but can additionally contain commonly known adhesives, adhesive proteins other than the adhesive proteins of the present invention, resin contained in commonly known coating agents, organic solvents, surfactants, anticorrosive agents, or pigments. The content of the additional components may be appropriately adjusted within the commonly accepted range depending on the kind of component and formulation of the coating agent. Where an additional component is included, the adhesive protein as an active component is contained in the coating agent at a level that maintains the adhesive activity, and can for example be contained in the coating agent at 0.1 to 80% by weight.

The coating agent of the present invention can be manufactured in the form of cream, aerosol (spray), solid, liquid, or emulsion, but is not limited to these formulations.

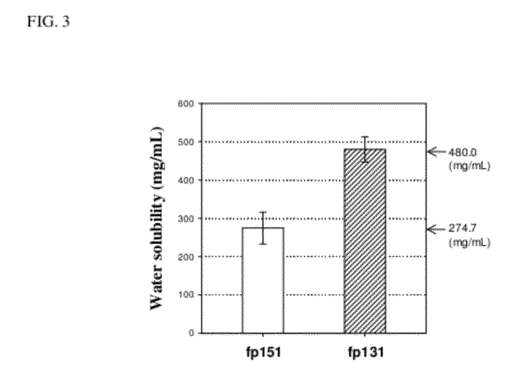

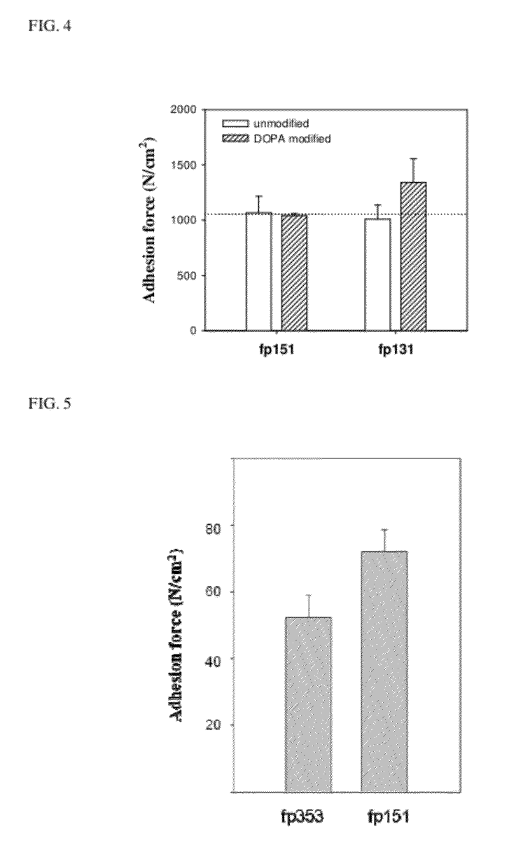

The fp-131 provided in the present invention shows higher expression level, protein production yield, and solubility than the known mussel adhesive proteins, fp-3 variant A and fp-353 (Table 1). As compared to fp-151 that is currently used as a hybrid mussel adhesive protein, it also shows approximately 1.8 times higher solubility in water (FIG. 3), and more excellent bulk adhesion force (FIG. 4). In addition, fp-151 having a lower bulk adhesion force than fp-131 showed approximately 1.3 times higher adhesion force than fp-353, suggesting that fp-131 shows a higher adhesion force than fp-353 (FIG. 5). Therefore, fp-131 of the present invention can be efficiently used in protein coating, cell adhesion experiment, or coacervation as an alternative to the known hybrid mussel adhesive proteins.

Hereinafter, the present invention will be described in more detail with reference to Examples. However, these Examples are for illustrative purposes only, and the invention is not intended to be limited by these Examples.

### Example 1

### Preparation of Mussel Adhesive Protein fp-151

The mussel adhesive protein fp-151 is composed of six fp-1 decapeptide repeats at both termini of fp-5, as described in WO2005/092920, the disclosure of which is incorporated herein by reference in its entirety. In the present invention, fp-151 gene was synthesized by GenScript Corporation (Centennial Ave., Piscataway, N.J. 08854, U.S.) with codon optimization for expression in host cells. The codon-optimized fp-151 was named fp-151-3.2, and the nucleotide sequences of the codon-optimized fp-151 is shown in SEQ ID NO: 12. The fp-151-3.2 was inserted into a pET22b(+) using NdeI and XhoI, so as to construct a pFP151-3.2.

### Example 2

### Preparation of Mussel Adhesive Proteins fp-3 and fp-353

2-1. Preparation of Mussel Adhesive Protein fp-3 Variant A

The mussel adhesive protein fp-3 variant A (SEQ ID NO: 9) was prepared by modification of an adhesive protein fp-3A (Genbank No. BAB16314 or AB049579) that is derived from one of the mussels, Mytilus galloprovincialis, and designated as Mgfp-3A MUTANT in WO2006/1071831. The preparation method thereof is the same as in the above patent literature, the disclosure of which is incorporated herein by reference in its entirety.

Specifically, the fp-3 variant A gene was cloned to construct a pMDG03 vector containing the fp-3 variant A gene according to the methods of Examples 1 and 2 described in WO2006/1071831, and E. coli BL21 was transformed with the pMDG03 vector to prepare and culture a transformant E. coli BL21/pMDG03 according to the method of Example 4 described in WO2006/1071831, and the fp-3 variant A protein was expressed and purified from the transformant E. coli BL21/pMDG03 according to the method of Example 5 described in WO2006/1071831.

2-2. Preparation of Mussel Adhesive Protein fp-353

The mussel adhesive protein fp-353 (SEQ ID NO: 14) was produced in E. coli by inserting the fp-5 gene (Genbank No. AAS00463 or AY521220) between two fp-3 variant A genes. The preparation method of the mussel adhesive protein fp-353 is the same as in WO2006/1071831, the disclosure of which is incorporated herein by reference in its entirety.

Specifically, the fp-5 gene and the fp-3 variant A gene were amplified using the pMDG05 vector containing the fp-5 gene described in Example 1 and the pMDG03 vector containing the fp-3 variant A gene described in Example 2-1, respectively and thus a pENG353 vector capable of producing a hybrid mussel adhesive protein fp-353 was prepared. The preparation method of the pENG353 vector was performed in the same manner as in Example 3 of WO2006/1071831. Next, E. coli BL21(DE3) (Novagen, USA) was transformed with the pENG353 vector to prepare and culture a transformant E. coli BL21(DE3)/pENG353 according to the method of Example 4 described in WO2006/1071831, and the fp-353 protein was expressed and purified from the transformant E. coli BL21(DE3)/pENG353 according to the method of Example 6 described in WO2006/1071831.

### Example 3

### Preparation of Mussel Adhesive Protein fp-131

The mussel adhesive protein fp-131 (SEQ ID NO: 8) was produced in E. coli by inserting the mussel adhesive protein fp-3 variant A gene between two fp-1 variant genes. The preparation method of fp-131 may be performed with reference to WO2005/092920 and WO2006/1071831, the disclosure of which is incorporated herein by reference in its entirety.

6×AKPSYPPTYK was attached to both the N- and C-termini of fp-3 variant A, so as to prepare a hybrid fp-131. Specifically, fp-151-3.2, a condon-optimized fp151, was inserted into a pET22b(+) using NdeI and XhoI, so as to construct a pFP151-3.2. fp1F in the pFP151-3.2 was amplified using a set of primers of SEQ ID NOs: 1 and 2, digested with NdeI/NcoI, and inserted into pET22b(+) digested with the same restriction enzymes so as to construct a pFP1F. Thereafter, the gene sequence of fp-3 variant A was amplified using a set of primers of SEQ ID NOs: 3 and 4, digested with NcoI/BamHI, and inserted into a pFP1F digested with the same restriction enzymes so as to construct a pFP13. Next, fp1R in the pFP151-3.2 was amplified using a set of primers of SEQ ID NOs: 5 and 6, digested with BamHI/XhoI, and inserted into pFP13 digested with the same restriction enzymes so as to construct a pFP131.

E. coli BL21(DE3) for protein expression was treated with CaCl₂ buffer to prepare competent cells, respectively. Each competent cell was transformed with pFP131 by heat shock (left at 42° C. for 2 minutes). Then, screening was performed using ampicillin (Sigma) to obtain E coli BL21/pFP131.

E. coli BL21/pFP131 was cultured in LB (5 g/liter yeast extract, 10 g/liter Tryptone and 10 g/liter NaCl) medium. For incubation experiment, E. coli was cultured in a 250 mL flask containing 50 mL LB medium, and the culture broth was inoculated in a 10 L fermenter containing 7 L of LB medium.

For protein expression, when absorbance of the culture broth reached 0.6 to 0.9 at 600 nm, IPTG was added at a final concentration of 1 mM to induce expression of the recombinant adhesive protein fp-131. After 6 hours, for isolation and purification of fp-131 protein produced in E. coli, disrupted cells were centrifuged at 6,000 rpm for 30 minutes, and TTE solution (1% triton x-100, 50 mM Tris-HCl (pH 8.0), 1 mM EDTA) with 50 mg/ml lysozyme was added to insoluble fraction for overnight at room temperature. After centrifuge at 9,000 rpm for 30 min, the cell pellet was washed twice with DW, and extracted with 25% (v/v) acetic acid solution. The extract solution was dialyzed with DW and freeze-dried.

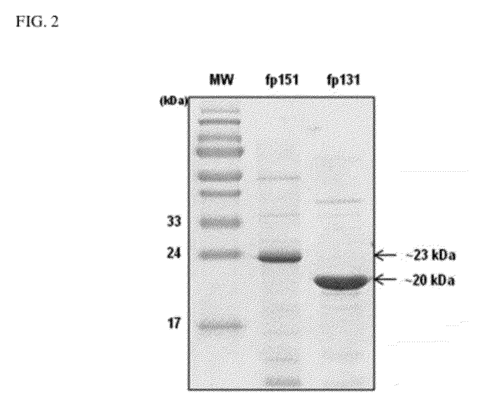

As a result, fp-151 showed a purity of approximately 89%, and fp-131 showed a purity of approximately 95% (FIG. 2).

### Example 4

### Modification of Tyrosine Residue of Adhesive Protein

Each of the fp-151, fp-3, fp-353 and fp-131 adhesive proteins prepared in Examples 1 to 3 was dissolved in a 5% acetic acid buffer solution containing 25 mM ascorbic acid at a concentration of 1.44 mg/ml, and then 50 ug/ml of tyrosinase was added thereto, followed by shaking at 25° C. for 6 hours. Through the above procedure, tyrosine residues of the adhesive proteins were modified to DOPA.

### Example 5

### Feature Comparison between fp-3 Variant A, fp-353 and fp-131

The pI value, protein expression yield, protein production yield, and solubility were compared between fp-131 of the present invention, and the known mussel adhesive proteins, fp-3 variant A and fp-353.

As a result, fp-131 showed higher expression yield and protein production yield than fp-3 variant A and fp-353. In particular, fp-3 variant A showed much lower protein expression yield and production yield. Thus, after isolation and purification, it was not suitable for bulk-scale test. In addition, when the protein solubility in a 5% acetic acid solution was compared, fp-131 showed more than 5 times higher solubility than fp-353. Therefore, fp-131 is advantageous over fp-353 in a high-concentration adhesion test.

TABLE 1

| | fp-3 | | |
|---|---|---|---|
| Feature | variant A | fp-353 | fp-131 |
| pI (calculated value) | 10.4 | 10.1 | 10.1 |
| Expression yield (%) | ~3 | ~21 | ~28 |
| based on total cellular | | | |
| proteins | | | |
| Production yield (mg/L) | ~3 | ~39 | ~40 |
| after purification | | | |
| Post-purification solubility | ~1 | ~90 | ~480 |
| (g/L) in 5% acetic acid | | | |

### Example 6

### Measurement of Solubility of Mussel Adhesive Proteins fp-151 and fp-131

In order to compare the protein solubility in a neutral solution, the freeze-dried mussel adhesive proteins fp-151 and fp-131 were dissolved in a 50 mM Tris-HCl (pH 6.2) buffer solution at a concentration of 500 mg/mL, and centrifuged at 13000 rpm for 30 minutes. Soluble proteins in the supernatant were quantified to analyze the solubility.

As a result, fp-131 showed approximately 1.8 times higher solubility than fp-151 (FIG. 3). Thus, fp-131 is advantageous over fp-151, when it is solubilized in water for practical use.

### Example 7

### Measurement of Adhesion Force of Mussel Adhesive Proteins fp-151 and fp-131

A bulk-scale adhesion force between fp-151 and fp-131 was compared in wood samples and leather samples. First, the freeze-dried single mussel adhesive protein fp-151 or fp-131 was dissolved in the same buffer, attached in the same manner, and dried at room temperature for 3 hours. A force was applied to both sides of the attached wood samples and leather samples, and shear strength was measured using a tensile strength tester (Instron) to measure the tensile strength of the adhesive protein.

When the bulk-scale adhesion force between fp-151 and fp-131 was compared in the wood samples, fp-151 and fp-131 showed similar adhesion force before DOPA modification, but fp-131 showed higher adhesion force than fp-151 after DOPA modification, indicating that tyrosine modification of fp-131 by tyrosinase occurred well due to its high solubility, leading to higher modification ratio (FIG. 4).

In addition, when the bulk-scale adhesion force between fp-151 and fp-353 was compared in the leather samples, fp-151 showed approximately 1.3 times higher adhesion force, suggesting that fp-131 showed higher adhesion force than fp-353 (FIG. 5).

What is claimed is:

1. An isolated adhesive protein comprising SEQ ID NO:8.

2. The adhesive protein of claim 1, wherein the adhesive protein further comprises a peptide for improving a physicochemical property selected from the group consisting of solubility, adhesion force, cross-linking, and improvement in protein expression, purification, recovery rate, and biodegradability of the adhesive protein, and the peptide is attached to a carboxy-termini or amino-termini of the adhesive protein comprising SEQ ID NO:8.

3. The adhesive protein of claim 2, wherein the peptide is isolated from an adhesive protein.

4. The adhesive protein of claim 3, wherein the adhesive protein is isolated from a mussel adhesive protein.

5. An adhesive comprising an adhesive protein according to claim 1 as an active component.

6. The adhesive of claim 5, wherein 5% to 100% of the total number of tyrosine residues in the adhesive protein is modified to 3,4-dihydroxyphenyl-L-alanine (DOPA).

7. The adhesive of claim 5, wherein the adhesive adheres to a substrate selected from the group consisting of plastic, glass, metal, eukaryotic cells, prokaryotic cells, plant cell walls and lipids.

8. The adhesive of claim 5, wherein the adhesive is applied to biological sample.

9. The adhesive of claim 5, wherein the adhesive further comprises one or more material selected from the group consisting of surfactant, oxidant, and filler.

10. The adhesive of claim 9, wherein the filler is selected from the group consisting of collagen, hyaluronic acid, condroitan sulfate, elastine, laminin, caseine, hydroxyapatite, and albumin, fibronectin, and hybrin.

11. The adhesive of claim 5, wherein the adhesive is applied to substrates used in an underwater environment.

12. A coating agent containing an adhesive protein according to claim 1 as an active component.

13. A polynucleotide comprising a nucleotide sequence encoding an isolated adhesive protein according to claim 1.

14. The polynucleotide of claim 13, wherein the nucleotide sequence encoding the adhesive protein comprises a nucleotide sequence comprising SEQ ID NO:7.

15. The polynucleotide of claim 13, wherein the nucleotide sequence encoding the adhesive protein further comprises a nucleotide sequence encoding a peptide for improving a physicochemical property selected from the group consisting of solubility, adhesion force, cross-linking, and improvement in protein expression, purification, recovery rate, and biodegradability of the adhesive protein, and the peptide is attached to a carboxy- and/or amino-termini of the adhesive protein comprising SEQ ID NO:8.

16. A vector that comprises a nucleotide sequence encoding an adhesive protein comprising according to claim 1.

17. A transformant transformed with the vector according to claim 16, wherein the transformant is selected from the group consisting of prokaryotes, eukaryotes, and eukaryote-derived cells.

18. The transformant of claim 17, wherein the eukaryote-derived cells are selected from the group consisting of plant cells, insect cells, and mammalian cells.

19. A method of producing an adhesive protein comprising the steps of:
(a) constructing a vector that comprises a nucleotide sequence encoding the adhesive protein comprising SEQ ID NO:8;
(b) constructing a transformant by transforming the vector into a host cell; and
(c) producing recombinant adhesive protein by culturing the transformant and purifying the recombinant adhesive protein.
